(19) United States
(12) Patent Application Publication
(10) Pub. No.: US 2011/0263937 A1
(43) Pub. Date: Oct. 27, 2011

(54) RIGID ENDOSCOPE

(75) Inventors: Eberhard Körner, Knittlingen (DE); Manfred BOEBEL, Bauschlott (DE); Waldemar SCHOLL, Ubstadt-Weiher (DE)

(73) Assignee: RICHARD WOLF GMBH, Knittlingen (DE)

(21) Appl. No.: 13/090,731

(22) Filed: Apr. 20, 2011

(30) Foreign Application Priority Data
Apr. 21, 2010 (DE) 10 2010 017 880.2

(51) Int. Cl. A61B 1/04; A61B 1/06

(52) U.S. Cl. 600/109; 600/160

(57) ABSTRACT

The rigid endoscope has a shank-like middle part, on which a handle connects on a proximal side and which on a distal side includes a distal part with an optics window. The handle is arranged offset or obliquely to a longitudinal axis of the middle part, as well as of the distal part, in order to provide free space in the region of the handle of the working instrument as well as on the distal side of the tool.

### BACKGROUND OF THE INVENTION

The present invention relates generally to a rigid endoscope and, more particularly, to a rigid endoscope having a shank-like middle part, onto which a handle part connects at a proximal side and which on the distal side includes a distal part with an optics window, with which the handle part is arranged offset or obliquely to a longitudinal axis of the middle part.

A rigid endoscope typically includes an optical system constructed exclusively of optical components forming a viewing window and electronic/optical systems forming the window for the optical beam path. Such rigid endoscopes may either be designed as pure endoscope optics or may be provided with one or more working channels/suction channels/rinsing channels, via which one or more instruments may be introduced.

Such a rigid endoscope in the form of a laparoscope is offered by Richard Wolf GmbH under the type number 8915.402. Such a rigid endoscope includes a handle part, on whose end an ocular of the endoscope optics is attached. The ocular is arranged obliquely to the longitudinal axis of the shank-like middle part of the instrument and thus leaves the access for a working channel running out in a flush manner to the middle part, with sufficient free space. It is also counted as belonging to the state of the art to manufacture the previously described rigid endoscope with a handle part which is arranged offset to the shank-like middle part, so that the view into the endoscope optics if not effected in the direction of the longitudinal axis of the shank-like middle part, but offset thereto.

With laparoscopic single-port technology, instead of several trocar sleeves, one operates with a single, specially adapted trocar sleeve. Not only optics, thus a rigid endoscope of the type being discussed, but additionally further working instruments are introduced into the abdominal cavity through this trocar sleeve. Thereby, it is of critical importance that the instruments thereby do not come into conflict with one another, by which means the work is made more difficult and for example one must operate in a crossed-over manner.

On the proximal side, this problem may basically be reduced by way of an extension in the grip region, which however not only makes the instruments unwieldy, but more susceptible to malfunction and more expensive. Flexible endoscopes provide remedy at the distal side. These provide significantly more degrees of freedom, but may not reduce the danger of conflict in the inside of the abdomen, since the distal endoscope end needs to be set obliquely with respect to the instruments. Added to this is the fact that such flexible endoscopes are significantly more complicated, liable to malfunction and more difficult in handling, than rigid endoscopes.

### BRIEF SUMMARY OF THE INVENTION

Against the above background, it is a objective of a preferred embodiment of the present invention to design an endoscope of the know type, such that in particular it may be advantageously applied for application in single port technology as well as with other endoscopic methods, and at least reduces the previously mentioned disadvantages and problems.

The rigid endoscope according to a preferred embodiment of the present invention includes a shank-like middle part, on which a handle part connects at the proximal side and which at the distal side comprises a distal part with an optics window, wherein the handle part is arranged offset or obliquely to the longitudinal axis of the middle part. According to a preferred embodiment of the present invention, the distal part is also arranged offset or obliquely to the longitudinal axis of the middle part. Advantageous designs of the present invention are specified in the subsequent description.

A basic concept of a preferred embodiment of the present invention is to arrange only the narrow, longitudinally extended shank-like middle part flush to the trocar sleeve, which forms the access into the abdominal cavity, and otherwise to arrange the handle part as well as the distal part offset to the longitudinal axis, so that not only is an instrument collision or a hindrance of the endoscopic instruments amongst one another avoided on the handle side, but also on the abdominal cavity side.

The rigid endoscope according to a preferred embodiment of the invention may thereby be designed either as singular optics or may also have one or more working channels/suction channels/rinsing channels. By way of the offset or oblique arrangement of the distal part, with regard to the design, one ensures that although on the one hand the viewing of the operation field within the body cavity and in particular also the distal end of the working instrument or instruments is not visible, the distal part itself however does not inhibit the handling of the working instrument or instruments.

It has been found to be particularly advantageous to arrange the handle part and the distal part in a flush manner with respect to the longitudinal axis of the middle part, for example to arrange the angular bend or the oblique setting in the same radial plane. A handling of such a designed endoscope in practice is particularly simple, since the inner-lying offset of the distal part or its oblique setting, which is not visible during the operation, is arranged exactly as the proximal visible part with regard to angle. This simplifies the spatial cognition during the operation.

Arranged obliquely to the longitudinal axis of the middle part in the context of the present invention is also to be understood as an arched arrangement, with which the distal part, proceeding from the middle part, not only firstly runs obliquely radially outwardly, but also in an arched manner and subsequently in its course runs inwards again or in a different suitable curve shape. Thereby, an arched course, with which the distal part is firstly directed outwards from the longitudinal axis of the shank-like middle part and then again is directed inwards, wherein however its ends at a distance to the longitudinal axis, has the advantage that one may use straight-viewing optics. Alternatively, the distal part may have an S-shaped or another curve course which is particularly useful for the respective operational region. Thereby, it is advantageous if the distal part or at least a section of this is likewise designed in a shank-like manner and in a manner such that it continues the shank-like middle part preferably with a constant cross section. An instrument which is particularly slim on the distal side then results and this may advantageously be provided with conventional optics, so that an optical connection exists between the distal part and the handle part.

Alternatively, the distal part may be designed in a widened manner with respect to the middle part, according to a further formation of the present invention and may have an electronic picture sensor which is optically connected to the optics window and whose electrical connections are led directly or indirectly to the handle part and are led out there. This design may be used in order to keep the middle part of the endoscope particularly slim, since then it is merely the cable lead-throughs and not an optical system, which run in this region. Then one must merely lead out the electrical connections at the handle part, either in the form of a connection plug or in the form of a fixedly connected electrical lead. Thereby, only the picture sensor may be arranged in the distal part, and the evaluation electronics in the handle part or outside the endoscope, and then the electronic picture sensor is led in a direct manner with its electric connections to the handle part and led out there. If, however, the evaluation electronics or a part of the evaluation electronics are arranged in the distal part together with the picture sensor, then the electrical connections of the picture sensor are led in an indirect manner, thus via the connections of the picture processing electronics, to the handle part and led out there. The picture sensor is optically connected to the optics window, in order in this manner, by way of the sensor which forms the part a video camera, to achieve an overview of the operation region within the cavity or of end effectors of the working instrument which are to be handled there.

Whereas with a purely optical design of the optical system on the handle part, typically an ocular and/or optics connection are provided, onto which for example a video camera head may be attached, with the design of the endoscope with an electronic picture sensor, the observation may be effected on the proximal side via an external screen.

The optics window of the endoscope according to a preferred embodiment of the present invention is advantageously provided at the distal end of the distal part and specifically preferably obliquely to the longitudinal axis of the distal part. The field of view, by way of the oblique arrangement of the window, may be directed in a targeted manner to the end effector region which is of interest here, without in particular a construction which is optically complicated with regard to design, being necessary. Alternatively, the optics window may also be arranged in a peripheral wall of the distal part. Such an arrangement is particularly useful if the distal part is arranged running obliquely outwardly with respect to the shank-like middle part.

Preferably, illumination means are integrated with the rigid endoscope according to a preferred embodiment of the present invention, preferably illumination means operating with at least one light diode. These may either be provided in the distal part or in the handle part. The arrangement in the distal part is particularly advantageous, since then no fiber-optics need to be led through the shank-like middle part.

According to an advantageous further formation of the endoscope according to a preferred embodiment of the present invention, the shank-like middle part does not have a round, but an oval cross section, in order to provide more space for the working instruments in the region of the trocar sleeve. Thereby, the cross-sectional arrangement is preferably of a type such that the offset or the oblique setting of the distal part and/or handle part are arranged in the direction of the short cross-sectional axis. The cross section thus with its large bending radius, thus with its flat side, bears on the trocar sleeve and projects significantly less into this compared to a round cross section.

### BRIEF DESCRIPTION OF THE SEVERAL VIEWS OF THE DRAWINGS

The foregoing summary, as well as the following detailed description of the present invention, will be better understood when read in conjunction with the appended drawings. For the purpose of illustrating the present invention, there are shown in the drawings preferred embodiments which are presently preferred. It should be understood, however, that the invention is not limited to the precise arrangements and instrumentalities shown. In the drawings:

FIG. 1 is a schematic representation of a trocar sleeve with a led-through, rigid endoscope, as well as with a working instrument, according to a preferred embodiment of the present invention,

FIGS. 2a-2c show three preferred embodiments of the distal part of the endoscope of FIG. 1,

FIGS. 3a and 3b show two preferred embodiments of a handle part of the endoscope of FIG. 1, and

FIGS. 4a and 4b show two preferred embodiments of a shank cross section of the endoscope of FIG. 1.

### DETAILED DESCRIPTION OF THE INVENTION

Certain terminology is used in the following description for convenience only and is not limiting. The words “proximal” and “distal” designate directions in the drawings to which reference is made. The words “inwardly” and “outwardly” refer to directions toward and away from, respectively, the geometric center of the device, and designated parts thereof, in accordance with the present invention. Unless specifically set forth herein, the terms “a,” “an” and “the” are not limited to one element, but instead should be read as meaning “at least one.” The terminology includes the words noted above, derivatives thereof and words of similar import.

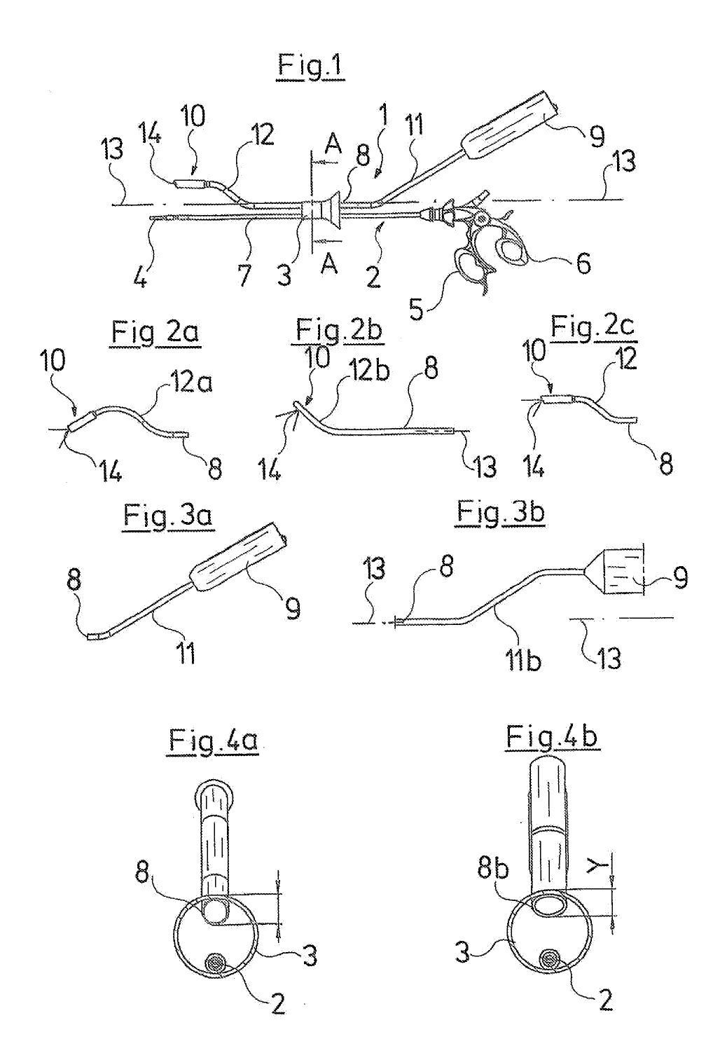

Referring to the drawings in detail, wherein like numerals indicate like elements throughout the several views, FIG. 1 shows a arrangement of a rigid endoscope 1 with a working instrument 2 within a trocar sleeve 3. The trocar sleeve 3, which maintains the access to the body cavity, is preferably designed in an essentially cylindrical manner and is widened on the proximal side in a funnel-like manner. Thereby, the region which is widened in a funnel-like manner lies outside the body interior, whereas the cylindrical region penetrates the skin and the abdominal wall and thus forms an access into the interior of the body.

With regard to the working instrument 2, it is the case of a forceps, whose distal-side end effectors 4 are to be handled by way of two grip parts 5, 6 arranged on the proximal side. The effectors 4 and the grip parts 5, 6 are connected via a straight shank part 7.

The endoscope 1 which is led through the trocar sleeve 3 parallel to the shank part 7, preferably includes shank-like middle part 8 which is designed in a straight-lined manner and is led roughly parallel to the shank part 7. On the proximal side, a handle part 9 connects to the shank-like middle part 8, and a distal part 10 connects to this at the distal side. Thereby, the handle part 9 is arranged obliquely to the longitudinal axis 13 of the shank-like middle part 8, and the distal part 10 is arranged offset thereto.

With the preferred embodiment according to FIG. 1, a proximal shank region 11 which is bent away outwardly in an oblique manner with respect to the shank-like middle part 8 connects to this middle part 8, via which shank region the actual handle part 9 connects, wherein this handle part typically has an ocular which is not shown, at its proximal end. A distal shank region 12 connects the shank-like middle part 8 to the actual distal part 10 which with the embodiment variants represented by way of FIGS. 1, 2a and 2c, is designed widened with respect to the shank region 12 and has an essentially cylindrical shape.

As is clearly shown in FIG. 1, the handle part 9 as well as the distal part 10 are arranged at a significant distance to the working instrument 2 in the respective regions, so that the instruments neither inhibit one another at the outside (proximal side) nor at the inside (on the distal side). The distal part 10, which is represented by way of FIG. 1, is connected to the shank-like middle part 8 via a distal shank region 12 which is offset in a parallel manner, so that the distal part 10 is arranged parallel and at a distance to the end effectors 4. The proximal shank region 11 in contrast is bent obliquely outwards with respect to the shank-like middle part 8, thus distances itself radially from the longitudinal axis 13 of the shank-like middle part 8 with an increasing radial distance to the trocar sleeve 3.

Alternative designs of the distal part 10 and of the distal shank region 12 are represented by way of the FIGS. 2a, 2b and 2c. With the embodiment variant according to FIG. 2a, the distal shank section 12a has a somewhat S-shaped course, so that the shank there, proceeding from the shank-like middle part 8, distances itself distally firstly from the longitudinal axis 13, in order then to be led back to this in a curve. The distal part 10 is arranged at the distal end of the shank region 12a and the optics window 14 of this distal part is directed in a direction which is oblique to the longitudinal axis 13 and thus permits an observation of the end effectors 4 or of the operational region which surrounds them.

With the embodiment according to FIG. 2b, the shank-like middle part 8 merges into the distal shank region 12b which runs in an oblique manner with respect to the longitudinal axis 13. The optics window 14 with this embodiment is arranged in the outer periphery of the distal shank region 12, which at the same time forms the distal part of the endoscope.

The embodiment variant of FIG. 1, with which the distal shank region 12 is designed in an angularly offset manner, i.e. crank-like manner and at the distal side receives a cylindrical distal part 10, on whose distal end the optics window 14 is arranged obliquely to the longitudinal axis of the distal part 10, in order in this manner to perceive the working region of the end effectors 4, is represented by way of FIG. 2c.

FIGS. 3a and 3b show in which way the proximal shank region 11 with the handle part 9 may be connected onto the shank-like middle part 8. The design according to FIG. 3a corresponds to that which is represented and described by way of FIG. 1. Alternatively to this, with the design according to FIG. 3b, the proximal shank region 11b is designed in an angularly offset manner, i.e. a crank-like manner, so that the handle part 9 is arranged parallel and offset to the longitudinal axis 13 of the shank-like middle part 8.

The handle part 9 is shown in the figures only by way of example by way of a cylinder-shaped body, but it is to be understood that this may also be formed by an optics connection, comprise an ocular, or may be designed in any other suitable manner. The shape represented here is only to represent one example for any common or suitable shape of a handle part of endoscope optics.

Two alternatives of the cross-sectional shape of the shank-like middle part 8 are represented by way of FIGS. 4a and 4b. The embodiment of FIG. 4a which shows a section of the trocar sleeve 3 in the region of the section line A-A represented in FIG. 1, illustrates that it is not only the trocar sleeve 3 which is circularly round, but also the shank-like middle part 8 in this region, as well as the working instrument 2 which likewise passes through the trocar sleeve 3. With the alternative embodiment according to FIG. 4b, the shank-like middle part 8b is designed in an oval manner and specifically in a manner such that the short axis characterized at Y in the figure, lies in the offset plane of the endoscope 1. This is defined in FIG. 1 by the plane of the paper. It is clearly visible in FIG. 1 that the distal part 10 as well as the handle part 9 are bent away or bent at an angle in the same plane with respect to the longitudinal axis 13 of the shank-like middle part 8. It is clear that with a shank design or shank arrangement as is represented by way of FIG. 4b, the usable free space within the trocar sleeve is significantly larger than that with a round design, as is represented by way of FIG. 4a.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-10. (canceled)

11. A rigid endoscope comprising a middle part (8) having a proximal side and a distal side, a handle part (9) connected to the proximal side and arranged offset or obliquely to a longitudinal axis (13) of the middle part (8), the distal side including a distal part (10) with an optics window (14), the distal part (10) arranged offset or obliquely to the longitudinal axis (13) of the middle part (8).

12. The endoscope according to claim 11, wherein the handle part (9) and the distal part (10) are arranged flush to one another with respect to the longitudinal axis (13) of the middle part (8).

13. The endoscope according to claim 11, wherein the distal part (10, 12a) is designed in an arched manner.

14. The endoscope according to claim 11, wherein the distal part (10, 12, 12a, 12b) and the middle part (8) are designed as a shank.

15. The endoscope according to claim 11, wherein an optics connection or an ocular is provided on the handle part (9) and the optics window (14) is optically connected, via an integrated optical system, to the optics connection or to the ocular.

16. The endoscope according to claim 11, wherein the distal part (10) is designed in a widened manner with respect to the middle part (8) and has an electronic picture sensor which is optically connected to the optics window (14) and whose electric connections are led directly or indirectly to the handle part (9) and are led out there.

17. The endoscope according to claim 11, wherein the optics window (14) is arranged at a distal end of the distal part (10) obliquely to a longitudinal axis of the distal part (10).

18. The endoscope according to claim 11, wherein the optics window (14) is arranged in a peripheral wall of the distal part (10, 12b).

19. The endoscope according to claim 11, wherein illumination means are provided in the distal part (10) or in the handle part (9) and operating with at least one light diode.

20. The endoscope according to claim 11, wherein the middle part (8b) has an oval cross section in a manner such that the offset or the oblique setting of the distal part (10) and of the handle part (9) is arranged in the direction of a short cross-sectional axis (Y).
